(19) United States
(12) Patent Application Publication
(10) Pub. No.: US 2013/0324857 A1
(43) Pub. Date: Dec. 5, 2013

(54) AUTOMATED SYSTEM FOR WORKSPACE, RANGE OF MOTION AND FUNCTIONAL ANALYSIS

(71) Applicant: THE REGENTS OF THE UNIVERSITY OF CALIFORNIA, Oakland, CA (US)

(72) Inventors: Gregorij Kurillo, Concord, CA (US); Jay Han, Folsom, CA (US); Richard Abresch, Davis, CA (US); Posu Yan, Berkeley, CA (US); Ruzena Bajcsy, Kensington, CA (US)

(21) Appl. No.: 13/831,608

(22) Filed: Mar. 15, 2013

Related U.S. Application Data
(60) Provisional application No. 61/653,922, filed on May 31, 2012.

(51) Int. Cl. A61B 5/11; A61B 5/00

(52) U.S. Cl. CPC A61B 5/1127; A61B 5/0013
USPC 600/476; 600/595

(57) ABSTRACT

A portable and automated system and method for measuring physical function utilizing a contactless and vision-based sensor system for acquiring human movements and methods for the analysis of reachable or functional workspace and range of motion that can be used in tele-medicine applications, such as remote functional assessment and diagnosis. An interactive, shared virtual 3D environment is provided where the patient can follow the movement directions provided by a remote physician while body kinematics are extracted from depth sensing cameras and wireless sensors.

### CROSS-REFERENCE TO RELATED APPLICATIONS

This application claims priority from U.S. provisional patent application Ser. No. 61/653,922 filed on May 31, 2012, incorporated herein by reference in its entirety.

### STATEMENT REGARDING FEDERALLY SPONSORED RESEARCH OR DEVELOPMENT

This invention was made with Government support under Grant Number H33B090001-10 awarded by the U.S. Department of Education. The Government has certain rights in the invention.

### INCORPORATION-BY-REFERENCE OF MATERIAL SUBMITTED ON A COMPACT DISC

Not Applicable

### BACKGROUND OF THE INVENTION

1. Field of Invention

This invention pertains to body motion assessment methods and systems and more particularly to a portable and automated system and method for the acquisition of human movement for functional workspace, range of motion, body segment movement and other goniometry assessments using a contactless vision-based camera/sensor system and optional wearable wireless sensors.

2. Background

Accurate and reliable assessments of body movements are critical for the diagnosis and characterization of various neuromuscular conditions and injuries, for tracking progress of therapy and to evaluate the effects of drug or surgical interventions. Traditional physical functional assessment of the body consists of measurements of range of motion (ROM), strength, endurance capacity, and reachable/functional workspace. There are many methods available to objectively quantify strength and aerobic work capacity. However, there is a lack of accurate, reliable, and practical methods to measure range of motion and reachable/functional workspace. Currently, measurements of limb range of motion rely on manual goniometry, which is quite subjective. These limitations largely prevent goniometry measurements to be clinically relevant or practical in closely monitoring a patient's function quantitatively or to be widely applicable and useful.

For the upper limb motion and function assessment, reachable/functional workspace measurements are closely associated with range of motion measurements. Current reachable/functional workspace determinations rely on manual goniometry measures that are not ideal and are fraught with problems in terms of useability, reliability, and accuracy or otherwise rely on cumbersome motion capture systems that require large spaces and expensive equipment.

Major components of a physical functional assessment of the body include range of motion, strength, endurance capacity, and reachable/functional workspace. There are various methods and technologies already available for strength measurements and aerobic work capacity measurements. These include manual muscle testing and quantitative strength measurement equipment such as isometric, isotonic, and isokinetic quantitative strength measure machines. Also included are measurements of activity and energy expenditure (oxygen consumption and carbon dioxide production monitoring machines, calorimeters, and step activity monitors), and validated methods/protocols for evaluating aerobic capacity for various physical activities and ambulation.

In contrast, there is no good range of motion measurement method that is user-friendly, accurate, reliable, or conveys overall functional capability of an individual in an intuitive 3-dimensional (3D) graphical manner. Traditional and current standards for goniometry measures for range of motion analysis for a particular body part is quite subjective, cumbersome, repetitive, tedious and time-consuming for a clinical evaluator, and often suffers from low accuracy and large inter- and intra-examiner variability. Part of the reason for this difficulty in manual goniometry is that the goniometry method inherently requires the isolation of an individual plane of joint movement with placement of the goniometer at specific locations, leading to variability due to evaluator experience as well as intra-examiner's measure to measure differences in the application of the goniometer itself. This difficulty in range of motion measurement is particularly problematic for a joint with multiple axes and/or rotational components to movement (e.g. shoulder, hip, ankle, spine, and neck). For the upper limb, the reachable/functional workspace concept is closely tied to range of motion of the upper limb. However, at this time reachable/functional workspace evaluation is also lacking practical and clinically useable technologies, as it either relies on inaccurate and unreliable manual goniometry data or requires cost prohibitive research infrastructure and space requirements with expensive specialized equipments. These limitations largely prevent current goniometry measures and reachable/functional workspace evaluations to be clinically relevant, practical, or widely used in monitoring patient function.

Accordingly, there is a need for accurate and reliable body range of motion measurements and reachable/functional workspace analysis that can play an important role in clinical practice and rehabilitation to evaluate the effects of surgical intervention, drug therapy and physical therapy. The present invention satisfies these needs as well as others and is generally an improvement over the art.

### SUMMARY OF THE INVENTION

The present invention generally provides a portable and automated system to measure physical function such as range of motion, body segment movements and reachable/functional workspace analysis using contactless vision-based camera sensor technologies and optional wearable wireless sensors with customized software algorithms. The framework is intended to be used with a variety of vision-based sensor/camera technologies (with or without additional wireless sensors). The invention is particularly suited for remote monitoring, functional assessment and diagnosis.

The focus of the system is on goniometry. Accordingly, the system measures body physical function including the range of motion at different joints (e.g. shoulder, elbow, wrist, neck, spine, hip, knee, and ankle) as well as reachable/functional workspace analysis (i.e. assessment of three-dimensional space a patient can reach with their hand or other joints/segments of the body). Instead of a long litany of joint angles as is currently done in the art, an intuitive 3D graphical visualization of the body segmental motion and function is created, and through its automatic detection and quantification of body segment movement, joint angle measurements as well as other parameterized body movements can be obtained.

The system is intended for use in clinical environments for the evaluation of physical function, the determination of disease/injury severity, the monitoring of disease/injury progression, rehabilitation monitoring, or for the evaluation of after applied treatment (e.g. surgery, medications, and therapies). For example, the system can be used in the clinical offices of orthopedic surgeons, rehabilitation specialists, sports medicine specialists, and primary care physicians, physical therapy and occupational therapy offices, patient home environments and research labs. The methods used are focused on the real-time assessment of parameters and therefore can also be used in real-time interactively to provide feedback to the patient as they perform the tasks and tests.

Given the cost-effectiveness of the vision-based detection system using a depth-sensing sensor or a stereo camera, components of the system or the whole system itself will have the ability to provide remote monitoring through telehealth/telemedicine applications. The data acquired from the depth-sensing camera, which includes the position and orientation of joints and/or body segments, color and 3D depth information, can be streamed through the network in real-time to facilitate remote assessment of the goniometry, reachable workspace or spatio-temporal trajectories. The real-time assessment and data obtained by the system allow for extension into virtual reality applications and this also opens up the potential use in telemedicine and tele-rehabilitation environments.

The relatively simple vision-based automated detection system using a depth-sensing camera (and optional wireless motion or other sensors, like electromyography (EMG)), has many advantages over current expensive and extensive motion capture systems and alternative systems that recognize gross movements and body segment motion. Additionally, the system is relatively inexpensive and portable, yet very accurate, quantifiable, and reliable, making it very translatable and appropriate for clinical settings. The system is comparable in accuracy and reliability to the full motion capture in a controlled laboratory setting. It also allows for flexibility in software for body recognition/tracking and potential use with telehealth/telemedicine applications and real-time, interactive, virtual reality and tele-immersion applications.

According to one aspect of the invention, a system and method are provided for the assessment and quantification of reachable workspace with a stereo camera, motion capture, or a depth-sensing camera.

According to another aspect of the invention, a method is provided for intuitive 3D graphical representations and reconstruction of upper extremity motion with automatic measurement and recording that can integrate with electronic health record systems.

Another aspect of the invention is a simplified upper limb and truncal movement protocol that is economical and fast but covers essentially the cardinal motions of shoulder and spine, as well as informing about functional capabilities about activities of daily living.

Another aspect of the invention is to allow the combination of the assessment with the use of weights (loading condition) to improve the sensitivity of the measurement.

A further aspect of the invention is to provide a system and method to evaluate body segments and produce a limb motion/functional assessment. For example, assessments of movement dysfunctions, spine range of motion, lower limb movement and function, transfer and ADL skill assessment (sit to stand, feeding, grooming, dressing, and toileting etc), as well as sitting position/posture can be conducted.

Another aspect of the invention is to provide a system and process for remote physical examination and assessment using depth-sensing cameras and wireless sensors that will allow physicians and therapists to remotely and quantitatively evaluate patients via the system.

Still another aspect of the invention is to provide a method for remote or local assessment of range of motion and workspace in conjunction with other wireless sensors for movement (such as accelerometers, magnetometers, and gyroscopes) and other physiological measures (such as surface electromyographic [EMG] sensor) to provide context-rich information about body movement that can be visualized with the movement results.

Another aspect of the invention is to provide a system and method for remote or local measurement of discrete path lengths of limb movement to measure and monitor tremor, smoothness, accuracy and trajectories of limb movement for the diagnosis, analysis and monitoring of neurological and movement disorders.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 is a flow diagram of a method for measured workspace, range of motion and functional analysis and tele-rehabilitation in a virtual environment according to one embodiment of the invention.

FIG. 2 is a three dimensional schematic diagram of trajectories of the hand and body parts captured during the performance of movement protocols.

FIG. 3 is a graph of hand trajectories of FIG. 2 transformed to body coordinates and fitted with a spherical workplace template according to the invention.

FIG. 4 is a graph of three-dimensional hand trajectory of FIG. 3 projected to spherical coordinates.

FIG. 5 is a graph of hand trajectories projected to spherical coordinates to obtain the outer boundaries of the concave bounding polygon using alpha shapes.

FIG. 6 is a graph plotting workplace template segmentation bounded by the measured trajectories with the surface area divided for each of the four quadrants for analysis.

FIG. 7 is a depiction of the analyzed workplace surface area calculated for each quadrant and normalized by the area of the hemisphere and quantified by other parameters.

FIG. 8 is a schematic framework for remote assessment of workspace and range of motion using depth sensing cameras according to one embodiment of the invention.

### DETAILED DESCRIPTION OF THE INVENTION

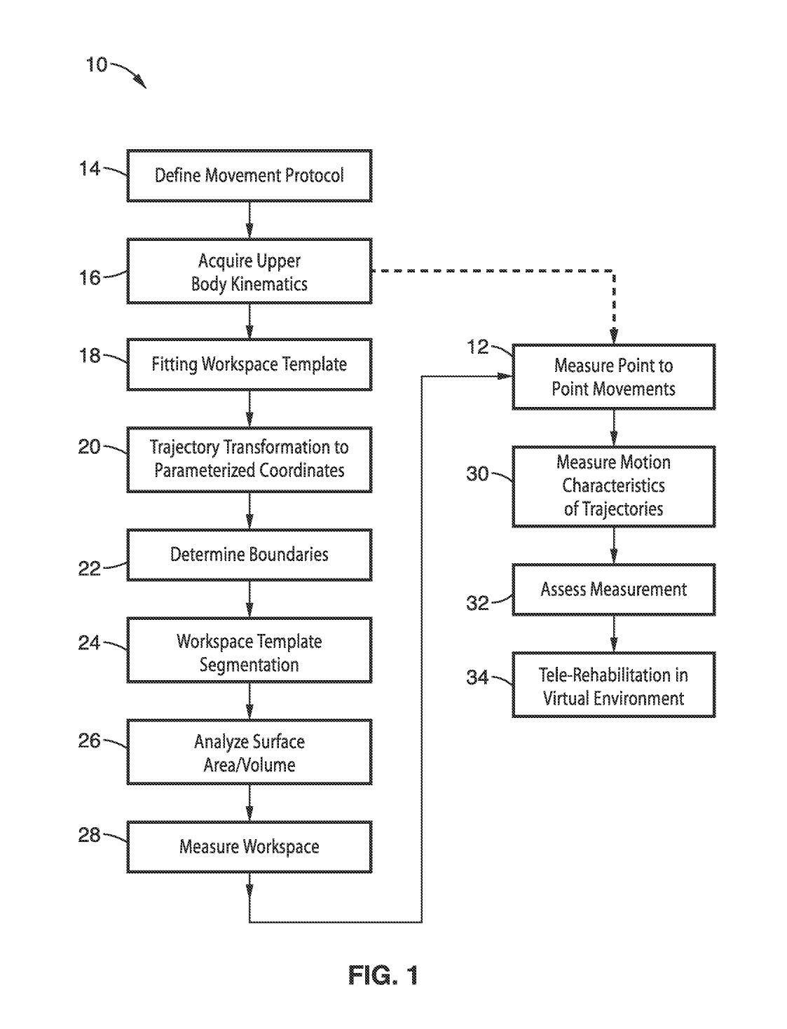

Referring more specifically to the drawings, for illustrative purposes several embodiments of the system and methods of the present invention are depicted generally in FIG. 1 through FIG. 8. It will be appreciated that the system and methods may vary as to the specific steps and sequence and the system architecture may vary as to structural details, without departing from the basic concepts as disclosed herein. The method steps are merely exemplary of the order that these steps may occur. The steps may occur in any order that is desired, such that it still performs the goals of the claimed invention.

By way of example, and not of limitation, FIG. 1 illustrates schematically a method 10 for measuring and imaging body motion and physical function utilizing a contactless and vision-based sensor system for the acquisition of human movement for the analysis of reachable or functional workspace, range of motion, and body segment movements. The methods and system can also be integrated into tele-medicine applications, such as remote functional assessment and diagnosis.

One embodiment of the method 10 for the measurement of reachable workspace using an unobtrusive, contactless, depth-sensing camera/sensor or other vision-based technology is illustrated in FIG. 1 with an upper extremity like the arm and hand. Generally, the camera captures the user's 3D information and extracts body kinematics (e.g. skeleton relating to the position of the joints) either directly from the range images or from markers placed on the body, such as in motion capture technology. The system with a single depth-sensing camera and optional sensor system significantly reduces the cost and space requirements compared with motion capture technology. The workspace analysis test is aimed at measuring the reachable workspace envelope of surface area as well as the reachable workspace volume, which will provide information on the functional abilities of a patient. Other properties of the workspace can also be analyzed, such as the shape of the workspace, for example. The reachable workspace can be divided vertically and horizontally with the shoulder joint as the origin, giving four wedge-shaped workspace or quadrants. For example, in the case of a patient with a neuromuscular disease, the observed muscle strength is gradually decreasing thereby reducing the ability of the patient to perform functional tasks in certain regions of their reachable workspace. Typically, the above the shoulder quadrants are affected initially. In addition, the assessment of the workspace can be performed under various load conditions where the limb of the patient is outfitted with a weight, securely attached at the endpoint. (e.g. a wrist weight). The data under different load conditions can imply the functional state of the patient's limb, such as in the case of the arm and shoulder as well as provide finer granularity with which to quantitatively measure an individual's functional capacity.

Measurement of point-to-point movements of the upper extremity during functional tasks and defined movements is conducted at block 12 from kinematic data from block 16 and processed workspace data from block 28. This task is focused on the measurement of the reachability of selected body points (e.g. mouth, ears, top of the head) that are related to the ability of performing functional tasks correlated to daily activities. The assessment is performed using motion tracking or another camera based system capturing the body position and limb endpoint location (e.g. hand) of the patient.

As seen in the method shown in FIG. 1, the vision-based 3-dimensional (3D) analysis of reachable workspace and range of motion measurements preferably begins with the general selection of the body areas and workspace context for evaluation. In the embodiment shown in FIG. 1, the upper torso and upper extremities are used to illustrate the system and methods. At block 14, one or more movement protocols are defined for the evaluation of the selected body area and workspace, such as a shoulder-based reachable workspace. The movement protocol is composed of a simple and economical set of movements for an upper limb that can provide essentially all of the information about the functional arm movements and normally does not require specific “targets.” However, the possibility of targets is not excluded.

Simplified upper limb movement protocols that are economical, efficient and fast but cover essentially the cardinal motions of shoulder, as well as informing about functional capabilities about activities of daily living are preferably defined at block 14. The shoulder range of motion protocol can be developed in conjunction with the software and made part of the software. The movement protocol is tailored for the vision-based sensor technology and for data collection (for acquisition of goniometry data as well as 3D reconstruction of reachable workspace and covering all four quadrants). Free-movement capture alone is not ideal because it lacks the standardization required for detection of important parameters, and may actually take longer to achieve all the cardinal motions if it is left to the individual to achieve all the necessary positions. This is because it will depend on the participant to eventually (and by chance) obtain the specific cardinal positions.

The set of movement protocols defined at block 14 preferably includes functional workspace movements that recapitulate some key elements found in activities of daily living (feeding, grooming, dressing, and toileting), as well as a reachable workspace movement protocol and separate shoulder internal and external rotation movements. The combination of functional workspace movements (that evaluate the range of motion for close-to-the-body activities, and provides the minimal workspace boundary) and the reachable workspace movement protocols (that evaluate the furthest reach of the hand, and provides the maximal workspace boundary) together allow for the calculation of reachable workspace volume that has not been achieved previously. For the body trunk and spine movement, a simplified protocol with hands at the hip with elbows flexed in its natural position can be used. Again, this and similar techniques will work in conjunction with the vision-based sensor system. The angled elbow essentially remains static in this position (regardless of torso movement) and as the trunk is flexed forward, extended backward, side-bend, and rotated, it can be detected. The movement protocol obviates the need for marker placement and standardizes the spine measurement, which is one of the difficult body segments for therapists or other clinicians to measure and quantify.

An example of a movement protocol for the evaluation of shoulder-based reachable workspace includes vertical sweep movements, horizontal sweep movements and shoulder rotation movements. Vertical sweep movements may be illustrated by: (Azimuth: 0 deg., Altitude: 0 to 180 deg.); (Azimuth: 45 deg., Altitude: 0 to 180 deg.); (Azimuth: 90 deg., Altitude: 0 to 180 deg.) and (Azimuth: 135 deg., Altitude: 0 to 180 deg.) movements. Horizontal sweep movements may be illustrated by: (Azimuth: 0 to 135 deg., Altitude: 30); (Azimuth: 0 to 135 deg., Altitude: 90 deg.); (Azimuth: 0 to 135 deg., Altitude: 0 to 180 deg.) and (Azimuth: 90 deg., Altitude: 0 to −90 deg.) movements.

The reachable workspace is defined by a set of all the points relative to the torso that an individual can reach by moving their hands, for example. The workspace envelope can be characterized by the encompassing surface area. It is not practical or feasible to ask the subject to reach all of the possible points. Therefore, the defined movement protocol leads the patient to perform movements in various standardized body planes. The obtained trajectory is then used to approximate the reachable workspace.

At block 16 the upper body kinematics of the patient are acquired using vision based cameras and optional sensors. A depth-sensing camera is applied to capture the three-dimensional position of the body via tracking or markers attached to the anatomical landmarks on the skin of the patient. In the case of marker based tracking, the software processing consists of several steps including marker detection and tracking, 3D triangulation and workspace analysis. The marker detection is performed via thresholding of a background-subtracted image, while searching for a circular-shaped marker within specific radius range. The markers are classified based on the size, location and color. Using Kalman and condensation filtering, the markers are tracked over time. For each tracker, the corresponding marker is determined from a combination of Euclidian distance and color similarity. For all candidates, probabilities are determined and the marker that has the highest probability is selected as the next tracker position. The tracking algorithm, based on these well-established methods, can deal with short occlusions and marker path crossings. In addition, the robustness of the tracking can be increased by using markers of different colors. Finally, the location of the trackers detected independently in the left and right image of the stereo camera is used in the triangulation calculation to determine its 3D position. Tests with the motion capture system have shown that achievable accuracy is in the range of 2-3 cm for the z-range. All these steps are performed in real-time to provide the assessor with feedback on the measurement process.

The utility of the process and principles for 3D motion analysis in the system can be illustrated by the fact that different vision-based technologies can be used to provide similar data (motion capture system, stereo-camera, or depth-ranging sensor such as Kinect). The invented process and principles for 3D motion analysis are not device-limited, and that is illustrated by the fact that the application of the sensor data derived from various vision-based technologies provide similar output results.

The body motion kinematics that are acquired at block 16 can be used to assist in the measurement of point-to-point movements at block 12 as well as in the characterization of the reachable workspace at block 18 through block 28 of FIG. 1.

When using a depth-sensing camera with body tracking capabilities or a motion capture system at block 16, the body kinematics are preferably obtained via a skeleton in each frame and directly used by the workspace analysis algorithm. The analysis of the workspace envelope may be performed offline. The tracked 3D hand trajectory that is transformed into a body-centric coordinate system is used to describe the outer envelope of the reached volume.

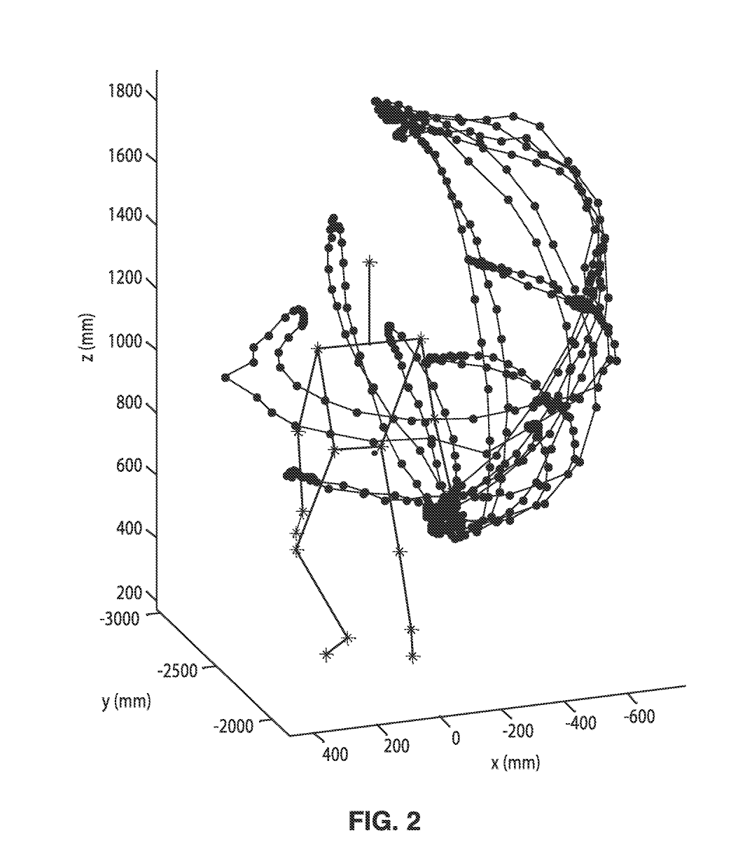

For the analysis of reachable workspace, the steps of block 18 through 28 are preferably used. The trajectory of the hand and body parts is captured during the performance of the prescribed movement protocol at block 16. One example of a trajectory capture is illustrated in FIG. 2.

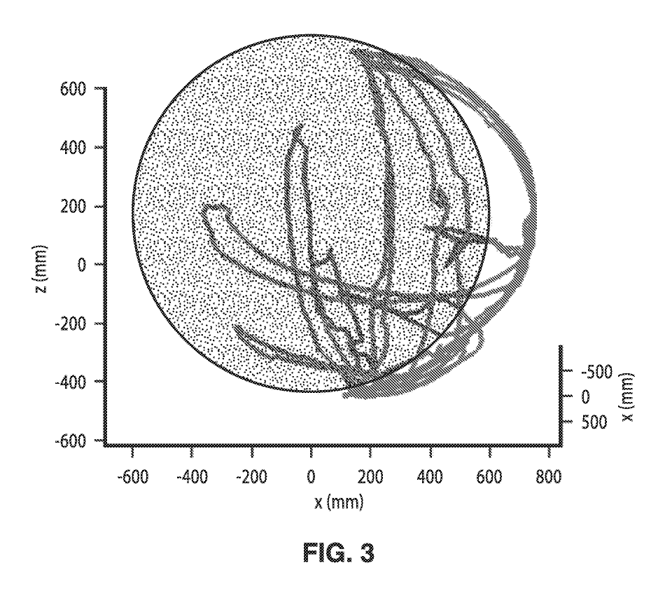

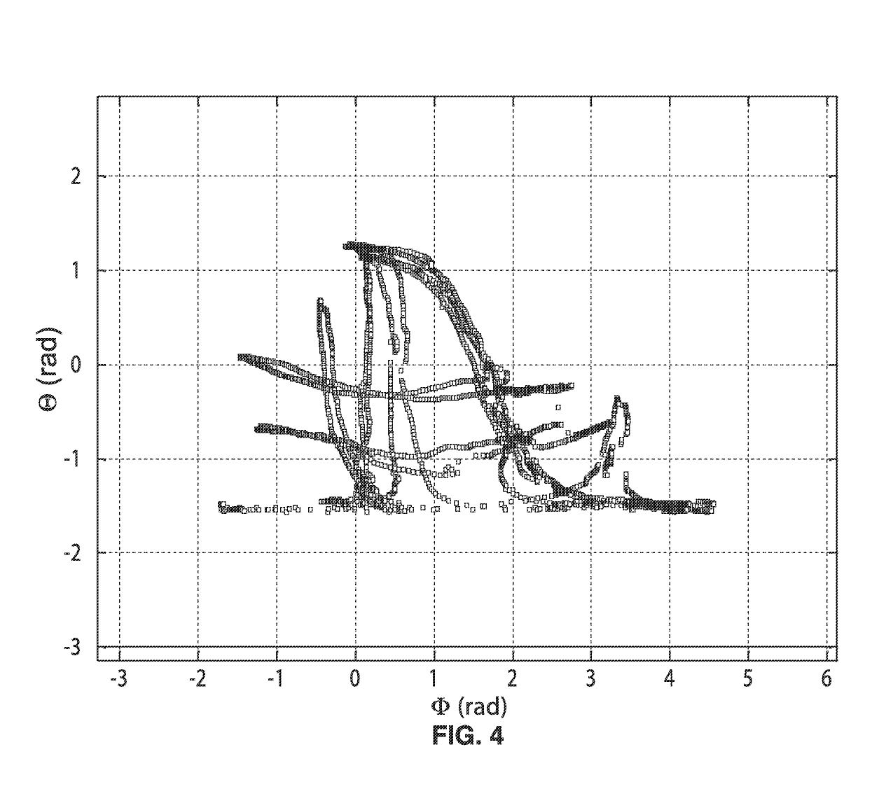

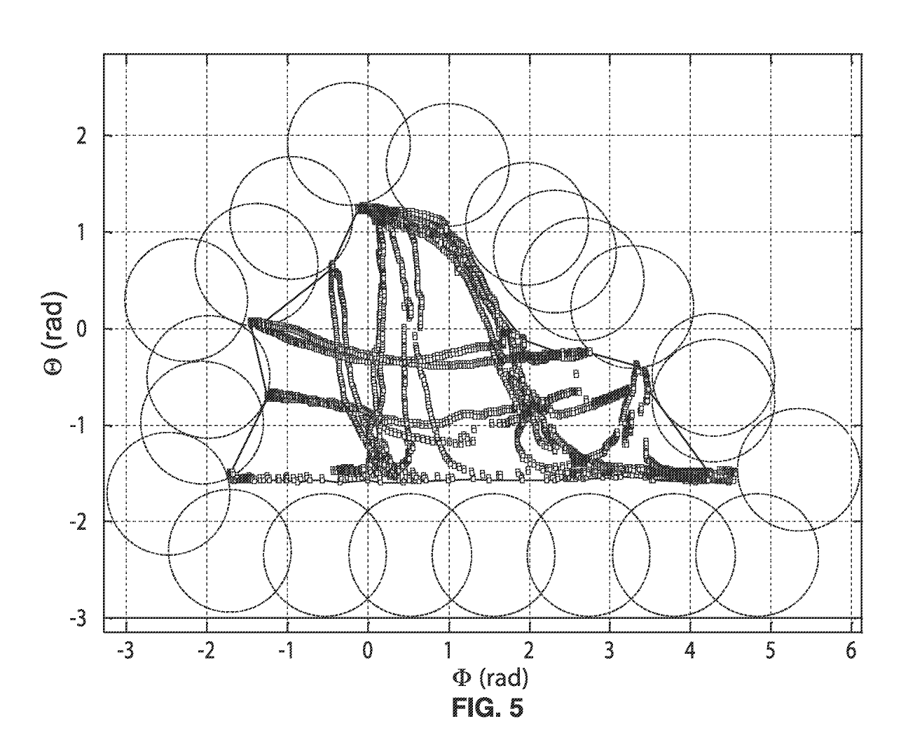

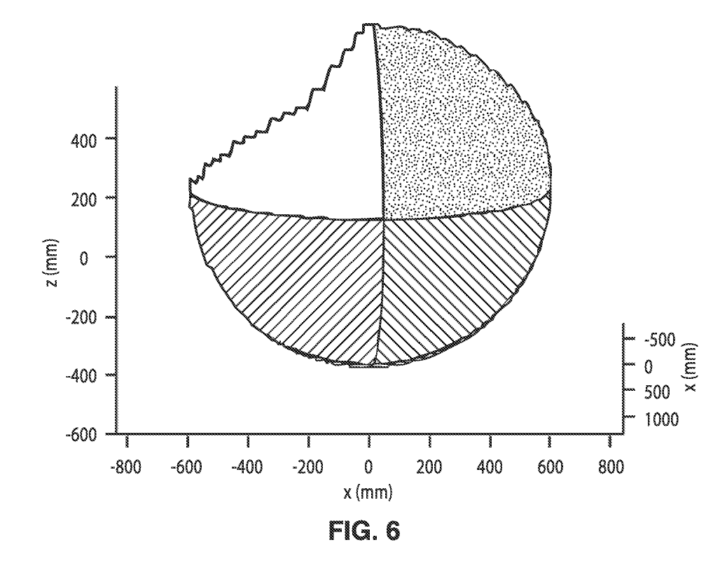

At block 18, the hand trajectory is transformed to body coordinates and fitted to a spherical or other surface that matches the expected workplace template as illustrated in FIG. 3. The hand trajectory points are then projected into spherical coordinates at block 20. An example of this projection is shown in FIG. 4. The bounding area is determined by fitting a concave polygon to the trajectory points at block 22. The concave polygon represents the boundaries of the carved workspace as seen in FIG. 5. Using previously determined boundaries, at block 24 the workplace template is culled to the surface that is bounded by the measured trajectory and the workplace template is segmented as illustrated in FIG. 6.

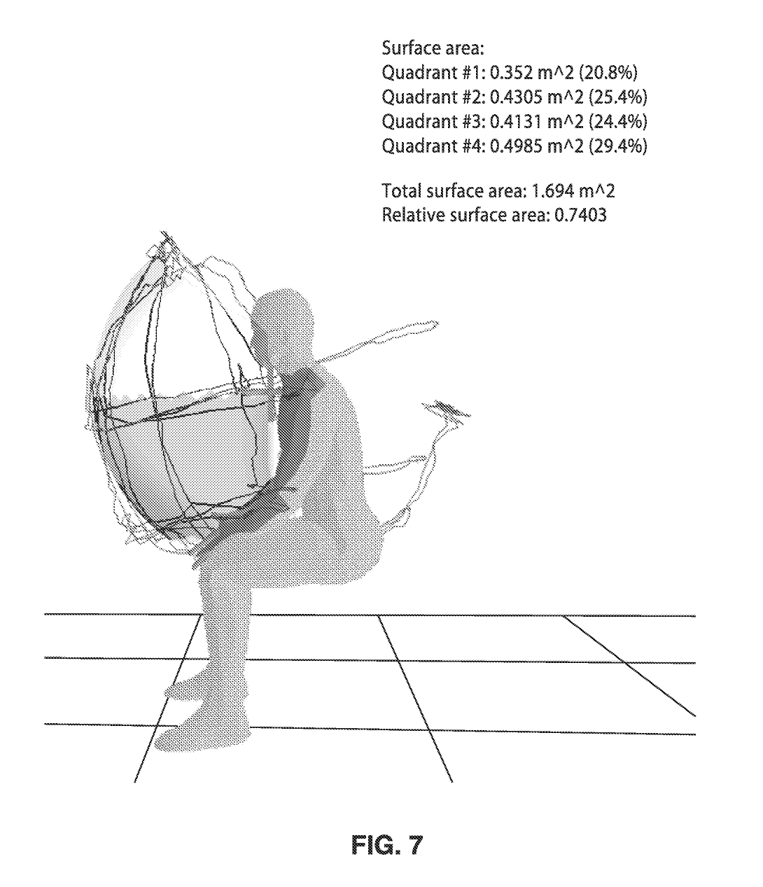

At block 26, the surface area and volume are analyzed. As shown in FIG. 7, the surface area is calculated for each quadrant and normalized by the area of the hemisphere (2πR²). Other parameters, such as workspace volume, deviation from the template, difference in volumes or surface areas are used to quantify the result. The workplace is measured at block 28 and all of the results are used to measure point-to-point movements at block 12.

To summarize the steps at block 18-28 and illustrated in FIG. 2 through FIG. 7, the trajectory points from block 16 are projected into a spherical coordinate system to parameterize the trajectory with two angles which also correspond to shoulder flexion/extension and abduction/adduction measurements in goniometry. In the parameterized space, it is possible to determine the maximal boundaries of the trajectory by fitting a concave polygon surface. The polygon is back-projected to the Cartesian coordinates to cull the template surface area (e.g. a sphere) which serves as an approximation of the unrestricted shoulder movement, for example. If the shoulder joint movement is approximated by a spherical joint, the boundary of the reachable space will be on a spherical/ellipsoidal surface. This is a reasonable approximation since the skeleton model only provides a simple kinematic chain of the body segments. Furthermore, the workspace area can be divided into quadrants that correspond to clinically significant functional subspaces, e.g. above/below shoulder, ipsilateral/contralateral side of the body with shoulder as the origin, in the sagittal plane.

To analyze the volume, the trajectory data is meshed to obtain a convex hull. The mesh data is now split into four quadrants with respect to the standardized human body planes. The sagittal plane defines the left and right side of the workspace and the horizontal plane defines the top and bottom part of the workspace. Each quadrant is then analyzed using alpha-shapes and the corresponding volume is calculated. Furthermore, the surface area or volume enclosed by the outer envelope of the reachable volume can be analyzed. The movement pattern can also be analyzed for the difference between the maximal reachable position/angle and the expected position/angle.

Optionally, at block 30 additional motion characteristics can be measured. Recording of body segment's static position, as well as various motion characteristics (length of trajectories, dynamics of movement, smoothness, and change-of-direction) can be conducted. Quantitative parameterized data can be collected that expands the utility of the process. For example, while performing the upper extremity reachable workspace evaluation, quantitative measurement of spinal and trunk position/posture can be achieved.

In addition, summation of various body segment path lengths of movement at each change of direction can provide a novel quantitative measure for tremor, ataxia (poor coordination), and movement dysfunctions. The sensitivity of the detection of movement disorder, in combination with the system, will also depend on the limitations imposed by the sensor hardware and the frequency with which the movement data is collected. However, as the vision-based technology sensors improve in resolution and frequency of data collection/sampling (frames per second, fps), the process and system framework can be used to improve the detection of various motion parameters.

Finally, all of the assessed measurements that have been taken can be collected, recorded, displayed and verified at block 32 of FIG. 1.

At block 34, the system can provide the basis for the tele-rehabilitation framework aimed for the remote assessment of range of motion and workspace and training of movement. Using wireless sensors, such as accelerometers, magnetometers, gyroscopes, and electromyographical sensors, for real-time sensing in connection with stereo and/or other depth-ranging sensors, the method can be used for remote evaluation and rehabilitation of a patient's function.

The stereo/depth ranging sensor(s) provide visual feedback and spatial data on a patient's body movement through tracking technology while other sensory data (e.g. from wireless accelerometers) is combined with the kinematics and provided as a real-time visual feedback to the patient and to the physician. Such systems could either be used within the medical facility or remotely from home using cost efficient technologies, such as smart phones and Kinect cameras for depth sensing.

In one embodiment, a teleimmersion system is used which allows users to collaborate remotely by generating realistic 3D representations of users in real time and placing them inside a shared virtual space.

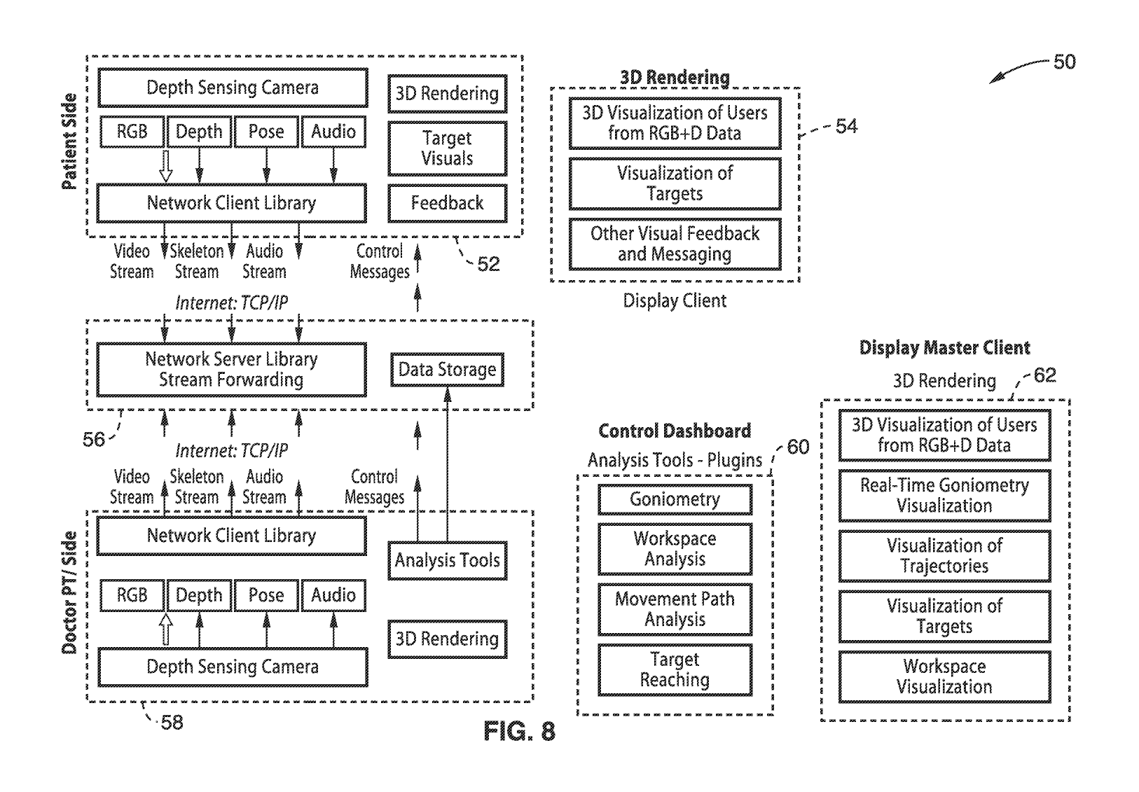

Referring also to FIG. 8, one embodiment of an interactive functional framework is depicted that can be used for remote assessments. Although the focus of the previous descriptions is on the upper extremities, it will be understood that the methods can be easily extended to other modalities and body functions. Data collected with the above described methods can be delivered in real-time to an integration module which can, in connection with stereo camera and/or other depth ranging sensors, provide visual feedback and spatial information on patient's movement.

In one embodiment, depth data is used to capture the 3D avatar of a user in real time and project it into a shared virtual environment, enabling a patient and therapist to interact remotely. Body tracking algorithms are applied to extract the kinematics data (i.e. joint location/angles) from the one or more spatial sensors. The marker based system can thus be replaced by the tracking algorithm. The 3D reconstruction can either be obtained from the stereo camera using the algorithm as described in FIG. 1, or from a commercial depth-ranging sensor such as Kinect. The sensor streaming framework allows for the connection of distributed virtual environments through the application of audio, video and other sensory data streams.

The system on the patient side shown in the top module of FIG. 8, for example, consists of the depth-sensing camera (with color sensor, depth sensor and microphone), large screen, computing system (e.g. desktop PC, laptop, tablet) with network connectivity. The patient sees on the screen as visual feedback of the 3D image of the doctor/PT generated by the remote depth-sensing camera. The 3D rendering is performed in the form of a mesh with texture map. Furthermore, the environment can also display simple targets (for reaching during the assessment) and feedback messages to the patient. The application, the display client, facilitates visualization for the patient but does not include any analysis tools.

The system on the doctor/PT side like that shown at the bottom of FIG. 8 includes similar depth-sensing cameras (with color sensor, depth sensors and microphone), large screen, computing system (e.g. desktop PC, laptop, tablet) and a tablet or “smart phone” based control system. The display shows the image of the patient generated by a 3D camera and the image is augmented with visual data received from the body tracking algorithm and/or wireless sensors. The doctor/PT can instruct the patient to perform certain movements by directly demonstrating them as the patient is able to see his or her image. The doctor/PT is also able to place various targets in the 3D environment that the patient may need to reach during the assessment. The interface is controlled via a tablet or smartphone controller that displays intuitively the assessment protocol and also provides doctor/PT the ability to control the application (e.g. data storage, calling different analysis plugins). The client application running on the doctor/PT side, i.e. Display Master Client, consists of the visualization with 3D rendering capabilities and a set of analysis tools (i.e. plugin tools) that can be loaded into the application on demand to perform specific analysis of the range of movement or other parameters related to the motion of the patient. The analyzed data is then sent to the server for storage and/or further analysis.

In the embodiment shown schematically in FIG. 8, the system 50 generally has three modules. The first module is the patient side module 52 with display client 54. The patient side module 52 has a depth sensing camera and subsystem for 3D rendering and for joint angle measurement, range of motion measurements and reachable space. The patient side module 52 also has a communications subsystem than is ultimately connected to the doctor/PT module 58.

The patient side module 52 is connected to a second module that is a network server module 56 via an Internet connection in the embodiment shown in FIG. 8. However, in other embodiments, the patient side module 52 can be connected to the doctor/PT module directly. The network module 58 is configured for data storage and network streaming between the patient side module 52 and the doctor/PT module 58.

The doctor/PT side module 58 also includes a control dashboard 60 with analysis tools and a master client display 62 for 3D rendering. The module has a depth sensing camera subsystem and a communications subsystem to provide a computer generated virtual environment in real time to allow remote assessment of range of motion and workspace etc.

The patient and doctor/PT communicate through a 3D collaborative environment that displays their 3D rendered real-time avatars generated by the depth-sensing sensor(s). The patient follows the movement directions provided by the remote assessor while the body kinematics is extracted from the depth sensing camera and/or wireless sensors. Data is displayed and analyzed on the assessors' side through overlaid real-time 3D visualization. The assessor can also provide the patient with virtual targets that the patient should follow. The dashboard on the assessor's side which can be controlled with a tablet that allows selection of analysis tools and display of data.

The invention may be better understood with reference to the accompanying examples, which are intended for purposes of illustration only and should not be construed as in any sense limiting the scope of the present invention as defined in the claims appended hereto.

### Example 1

In order to demonstrate the functionality of the motion capture system and assessment of the workspace envelope, a camera with full-body tracking capabilities, and a custom-designed 3D virtual environment for visual feedback and protocol for upper extremity evaluation were assembled. The Kinect camera that was selected captures depth and color images with 30 frames per second (fps), generating a cloud of three-dimensional points from an infrared pattern projected onto the scene. The resolution of the depth sensor is 320×240 pixels providing depth accuracy of about 10-40 mm in the range of 1-4 m. For validation of the Kinect skeleton tracking data, we simultaneously captured the subjects using commercial marker-based motion capture system Impulse (PhaseSpace, Inc., San Leandro, Calif.). Impulse motion capture system can uniquely identify and track 3D position of LED markers with the frequency of 480 Hz and sub-millimeter accuracy.

For the experiments, a tight-fitted shirt equipped with 18 markers was used. In addition, three markers were applied on the dorsal side of each hand and three markers on a tight-fitted cap to mark the top part of the head. In total 27 markers were used to capture the upper body. Since it is difficult to position markers on anatomical landmarks, a skeletonization method integrated in the software was used. For each subject we recorded a calibration procedure which involved exercising movement in the wrist, elbows and shoulder joints while keeping the rest of the body in a T-pose. From the calibration data, the algorithm determined location of the joints and is thus able to fit an anthropometric skeleton into the marker data. The Recap skeletonization algorithm is able to provide a skeleton even when some markers are occluded.

A simple set of movements were developed consisting of first lifting the arm from the resting position above the head while keeping the elbow extended, performing the same movements in the vertical planes of about 0, 45, 90, and 135 degrees. The second set of movements consists of horizontal sweeps at the level of umbilicus and shoulder. Both vertical and horizontal movements were performed in one recording session, lasting less than 1 minute. The movement protocol was developed and refined through a series of experiments with healthy persons and individuals with various forms of neuromuscular diseases.

To facilitate objective evaluation of the upper extremity reachable workspace, the users were presented with a visual feedback. The 3D environment featured the video of the therapist performing the protocol, and a mirrored 3D image of the user as captured by the Kinect camera. Visual feedback of the user was found to provide important visual cues for following the movement protocol. For the feedback we deliberately displayed only a texture-less 3D image since patients may not be comfortable watching a full (textured) video of themselves.

The validation of the Kinect-based reachable workspace evaluation was performed in ten (10) healthy subjects. Simultaneous recordings of the motion capture markers and Kinect skeleton data were collected. After donning the suit with markers, we collected calibration data for each subject. During the entire procedure the subject was seated on a chair and instructed to keep the back upright. Each subject first watched an instructional video in full screen mode. The kinesiologist in person provided additional instructions on body posture and limb positioning during various sequences of the task. Next, the subject performed three repetitions of the protocol on each side of the body while observing the visual feedback provided on a 55″ TV screen.

The reachable workspace is defined by a set of all the points relative to the torso that an individual can reach by moving their hands. The workplace envelope can be characterized by the encompassing surface area. It is not practical or feasible to ask the subject to reach all the possible points. Therefore the trajectory obtained from the movements in various standardized body planes is used. In 3D space, the obtained hand trajectory can be interpreted as a point cloud where the points lie on a surface of the reachable envelope of the arm. Since the arm trajectory covers only a portion of the space, it is not possible to determine the enclosed surface by a simple Delaunay triangulation. Instead, the shoulder joint movement is approximated by a spherical joint and parameterizes the trajectory in spherical coordinates with two angles corresponding to shoulder flexion/extension and abduction/adduction measurements in goniometry. This is a reasonable approximation since the skeleton model only provides a simple kinematic chain of the body segments.

After the mapping into the spherical coordinates, the boundaries of the trajectory were determined by a concave polygon. The polygon was determined by using the alpha shape with radius π/4 to tightly fit the data points. Finally the boundary of the polygon is projected back to the Cartesian coordinates to obtain their equivalent 3D trajectory. The resulting boundary lies on the spherical surface which can then be culled accordingly to retain only the surface inside the point cloud of hand positions. Furthermore we divide the workspace area into several quadrants that correspond to clinically significant functional subspaces, e.g. above/below shoulder, left/right side of the body. The sagittal plane divides the surface into left and right side of the workspace and the horizontal plane (at the level of the shoulder joint) divides the top and bottom part of the workspace. The reported surface area was calculated for the entire workspace envelope and for individual quadrants. To allow for comparison between different subjects, the absolute surface area is normalized as the portion of the unit hemi-sphere that is covered by the hand movement. It is determined by dividing the absolute area by the factor 2πR². The parameter R, which represents the average distance of the hand from the shoulder, is determined by the least-squares sphere fitting algorithm. The relative surface area of 1.0 would thus correspond to the entire frontal hemisphere that the subject could reach, with its origin in the shoulder joint.

### Example 2

To further demonstrate the function of the methods, a system to measure physical function utilizing contactless and vision-based sensor system for acquisition of human movement with customized software algorithms was provided for analysis of reachable or functional workspace and range of motion that can be used in tele-medicine applications, such as remote functional assessment and diagnosis.

A simple stereo camera-based reachable workspace acquisition system combined with customized 3D workspace analysis algorithm was developed and compared against a sub-millimeter motion capture system. The stereo camera-based system was robust, with minimal loss of data points, and with the average hand trajectory error of about 40 mm, which resulted to ˜5% error of the total arm distance. To demonstrate the system, a pilot study was undertaken with healthy individuals (n=20) and a select group of patients with various neuromuscular diseases and varying degrees of shoulder girdle weakness (n=9). The workspace envelope surface areas generated from the 3D hand trajectory captured by the stereo camera were compared. Normalization of acquired reachable workspace surface areas to the surface area of the unit hemi-sphere allowed comparison between subjects. The healthy group's relative surface areas were 0.618±0.09 and 0.552±0.092 (right and left), while the surface areas for the individuals with neuromuscular diseases ranged from 0.03 and 0.09 (the most severely affected individual) to 0.62 and 0.50 (very mildly affected individual). Neuromuscular patients with severe arm weakness demonstrated movement largely limited to the ipsilateral lower quadrant of their reachable workspace. The findings indicated that the stereo camera-based reachable workspace analysis system is capable of distinguishing individuals with varying degrees of proximal upper limb functional impairments.

To obtain the position of markers in 3D space, two geometrically calibrated cameras with time synchronization were used. For the measurements, a BumbleBee2 camera (Point Grey Inc., Richmond Canada) was used, which is a stereo camera with two imagers, each producing an image with the resolution of 1024×768 pixels at the frame rate of 20 FPS. The baseline of the camera, describing the distance between the two imagers, was 12 cm. The stereo camera was used in the clinical setting to track the location of different body landmarks marked with small LED markers.

Detection and labeling of markers from the images captured by the stereo camera were performed by the tracking algorithm. Data processing consists of the following steps: (1) marker detection, (2) marker tracking, (3) triangulation, and (4) workspace analysis. The marker detection from the images is performed via thresholding of the background-subtracted image, while searching for circular-shaped markers within specific radius range. The location of the marker center is determined by calculating the center of marker intensity with sub-pixel accuracy.

For motion data collection, five markers were tracked that were applied to the upper torso and abdomen (suprasternal notch, acromion process, and umbilicus) and the tip of the middle finger. For the body markers high luminance LEDs (Luxeon III, Phillips Lumiled) were used. For the hand, a white light source supplied by a pencil flashlight with diffuser removed to achieve highest level of visibility from any angle. The substitution of the marker color for the clinical experimental procedure did not affect the accuracy of the marker detection algorithm since the center of the marker was calculated from the intensity (grayscale) image.

Anthropometric measurements of arm length were obtained for each subject (distance between the acromion process LED and tip of middle finger where the white light marker was located). Subjects were seated in a chair, located about 2 m from the camera, with their arms at their sides (which was designated as the starting position, or the neutral position). The chairs had no arm supports or arm rests. The impaired individuals who were in a wheelchair performed the experiment from the wheelchair with the arm rests removed. A strap was applied below the axilla to minimize the movement of the trunk during the measurements. Markers were applied to the skin using simple Velcro adhesive tapes. The subjects were then shown the study protocol movements by the study kinesiologist and instructed to mirror the movements. A standardized simple set of movements consisted of lifting the arm from the resting position to above the head while keeping the elbow extended, performing the same movement in vertical planes at around 0, 45, 90, 135 degrees. The second set of movements consisted of horizontal sweeps at the level of the umbilicus and shoulder. The entire sequence of movements was recorded together. The study protocol movements were simple to perform for the subjects and typically took less than 1 minute for the entire sequence of movements. The shoulder underwent its full ROM (except for the extreme shoulder extension that is limited by the back of the chair). Each set of movements was repeated three times for left and right arm. Subjects were instructed to reach as far as they can while keeping the elbow straight. If they were unable to reach further, they were to return to the initial position and perform the next movement. During the measurements a kinesiologist demonstrated the movements in front of the subject to dictate the speed and order of movement segments, and if the subject leaned or trunk rotations were observed by the kinesiologist, the recording was repeated from the beginning. A total of 20 healthy individuals (12 female, 8 male; average age: 36.6±13.6 years) and 9 patients (all male but one, average age: 46.2±16.3 years) with various neuromuscular conditions participated in the study.

The analysis of the workspace envelope was performed offline. The tracked 3D hand trajectory was first transformed into body-centric coordinate system defined by the four markers on the body. The data was filtered with 3^{rd} order Butterworth filter with the cut-off frequency of 10 Hz. Large outliers (i.e. spikes) due to triangulation error were removed using an implementation of phase-space despiking. In 3D space, the obtained hand trajectory was interpreted as a point cloud where the points lie on a surface of the reachable envelope of the arm. To simplify the analysis we fitted a spherical surface into the data points. Due to noise and the simplification of the shoulder joint, some of the points were offset from the surface, however, the errors were in the order of a few centimeters. To obtain the boundaries of the surface, the data was first transformed into spherical coordinates by projecting the points close to the sphere onto the surface of the sphere and eliminating outlying points. Since the radius was fixed, the projected data was two-dimensional and parameterized with the corresponding vertical and horizontal angles. The boundary points were obtained using alpha shape. Alpha shape consists of piece-wise linear curves which approximate a concave surface containing the set of points. The level of concavity is defined by the circumscribed circle defined along the convex boundary (circle radius was π/4). The spherical surface represented by small rectangular patches (i.e. quads) was segmented using the boundary curve of the alpha shape. The quads were culled depending whether their centers lie within the alpha shape in the spherical coordinates or not. Furthermore, the surface data was split into four quadrants corresponding to the coordinate system placed in the shoulder joint and defined by the standardized human body planes. The sagittal plane defined the left and right side of the workspace and the horizontal plane (at the level of the shoulder joint) defined the top and bottom part of the workspace.

The reachable surface area was calculated for each of the quadrants and the summated total area, as well as the relative surface area. The relative surface values are reported as a percentage of the total surface area. The surface area was normalized with respect to surface area of a unit hemi-sphere (with radius 1.0) to be able to compare the results between subjects. The assessed relative surface area therefore lies between 0.0 and 1.0, where 1.0 represents reachable workspace envelope of the entire (frontal) hemi-sphere.

The 3D hand trajectory with fitted 3D surface for healthy subjects and the various patients were evaluated. The 3D surface area was divided and analyzed for each of the four quadrants. The control data of the healthy subjects had a quite equal distribution of surface area between the top and bottom quadrants. The patient with Becker Muscular Dystrophy (BMD) produced similar movement with somewhat reduced reachability at the top of the quadrants. The patient with Duchene Muscular Dystrophy (DMD) was able to perform movement primarily in the lateral coronal and sagittal planes but lacked the strength to raise the arm in the other directions. The results of the two patients with Facioscapulohumeral muscular dystrophy (FSHD) represent the wide range of performance of patients with the shoulder weakness. One patient was able to move into all four quadrants, while the other patient only produced movement in the lower ipsilateral quadrant due to the muscular weakness. Finally, a patient with relatively advanced Pompe disease was also able to move his hand only in the lower ipsilateral quadrant resulting in small overall surface area. The difference in 3D reachable workspace and abstracted upper limb functional status can be readily visualized between a healthy individual and individuals with varying degrees of shoulder girdle muscle weakness due to neuromuscular disorders.

The relative surface areas of the reachable envelope in the healthy controls and individuals with various neuromuscular diseases resulting in upper limb weakness were also evaluated. The relative surface area represents the portion of the unit hemi-sphere that was covered by the hand movement. It is determined by dividing the area by the factor 2πr², where r represents the distance between the shoulder and fingertips. This allows scaling of the data by each person's arm length to allow normalization for comparison between subjects. The subjects covered relative surface area of about 0.60 which corresponds to 60% of the surface area of the frontal hemi-sphere. The mean relative surface area in healthy persons was 0.618 (SD±0.080) for the right arm and 0.552 (SD±0.092) for the left arm.

Accordingly, the application of the developed 3D workspace acquisition system using a stereo-camera and a customized algorithm to determine the surface envelope area was demonstrated on actual individual patients with varying degrees of upper limb dysfunction due to neuromuscular diseases (Becker muscular dystrophy, Duchenne muscular dystrophy, Facioscapulohumeral muscular dystrophy and Pompe disease).

The results suggest that the developed methodology has adequate range of sensitivity to determine not only the healthy individuals from those with neuromuscular disorders, but also capable of separating out those with severe upper limb dysfunction from those with milder phenotypes. In patients with neuromuscular diseases there is a substantial need for quantitative assessment methods which could track progress of the disease or effects of novel treatment methods. Many of the functional tests are not specific enough for wide range of impairments resulting from neuromuscular diseases and they provide only qualitative assessment. The results suggest that the evaluation through reachable workspace envelope can provide quantitative information on ability to reach for objects with straightforward at-a-glance visualization of the overall functional capability of the upper limb. The results also suggest that similar methodology can be applied towards post-surgical patients as well as tracking therapeutic efficacy during physical therapy and pharmacologic treatments (in clinical setting and drug trials).

From the discussion above it will be appreciated that the invention can be embodied in various ways, including the following:

1. A system for measuring joint angles, range of motion, reachable space, and other motion characteristics of a person, the system comprising: (a) a plurality of markers for attachment to anatomical landmarks on a person; (b) a camera for capturing three-dimensional position of the markers; (c) a computer configured for acquiring images from the camera; and (d) programming executable on the computer and configured for marker detection, marker tracking, 3D triangulation and workspace analysis.

2. The system of any previous embodiment, wherein marker detection is performed by thresholding of a background-subtracted image while searching for a circular-shaped marker within a specific radius range.

3. The system of any previous embodiment, wherein the programming is further configured for classifying the markers based on size, location and color.

4. The system of any previous embodiment, wherein the programming is further configured for tracing the markers over time.

5. The system of any previous embodiment, wherein the programming is further configured for, for each tracker, determining the corresponding marker from a combination of Euclidian distance and color similarity; and for all candidates, determining probabilities selecting the marker with the highest probability as the next tracker position.

6. The system of any previous embodiment, wherein the programming is further configured for deterring 3D position from tracker location detected independently in left and right images of the stereo camera.

7. The system of any previous embodiment, wherein the programming is further configured for analyzing a workspace envelope.

8. The system of any previous embodiment, wherein the programming is further configured for transforming tracked 3D hand trajectory into a body-centric coordinate system.

9. The system of any previous embodiment, wherein the programming is further configured for using a body-centric coordinate system to describe the outer envelope of the reached volume.

10. The system of any previous embodiment, wherein the programming is further configured for meshing data to obtain a convex hull.

11. The system of any previous embodiment, wherein the programming is further configured for splitting the mesh data into four quadrants with respect to standardized human body planes.

12. The system of any previous embodiment, wherein the sagittal plane defines the left and right side of the workspace and the horizontal plane defines the top and bottom part of the workspace.

13. The system of any previous embodiment, wherein the programming is further configured for analyzing each quadrant using alpha-shapes and calculating corresponding volume.

14. The system of any previous embodiment, further comprising: a remote therapist module configured for communicating with the computer, the therapist module comprising: (a) a therapist computer with a display; (b) at least one camera operably coupled to the computer; (c) programming executable on the therapist computer configured for communicating with a patient computer, workspace analysis and image rendering.

15. The system of any previous embodiment, further comprising: a remote network server module configured for communicating with the computer and the therapist module, the remote network server module comprising: (a) a computer; and (b) programming executable on the therapist computer configured for communicating with a patient computer and a therapist computer and data storage.

16. A method for measuring joint angles, range of motion, reachable space, and other motion characteristics of a person, comprising: acquiring body part motion kinematics in three dimensions from a camera; measuring body part motion trajectories; and calculating reachable workspace envelope.

17. The method of any previous embodiment, further comprising: measuring discrete path lengths of body part movements.

18. A method for measuring joint angles, range of motion, reachable space, and other motion characteristics of a person, comprising: (a) defining a movement protocol for a body part; (b) capturing body part trajectories of a subject during performance of the movement protocol for the body part; (c) fitting trajectories to a workspace template; (d) transforming fitted trajectories to parameterized coordinates; (e) determining boundaries from coordinates; and (f) formulating reachable workspace envelope.

19. The method of any previous embodiment, further comprising segmenting the workspace template into segments.

20. The method of any previous embodiment, further comprising: calculating reachable workspace surface area; and calculating reachable workspace volume.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

We claim:

1. A system for measuring joint angles, range of motion, reachable space, and other motion characteristics of a person, the system comprising:
(a) a plurality of markers for attachment to anatomical landmarks on a person;
(b) a camera for capturing three-dimensional position of the markers;
(c) a computer configured for acquiring images from the camera; and
(d) programming executable on the computer configured for marker detection, marker tracking, 3D triangulation and workspace analysis.

2. The system of claim 1, wherein marker detection is performed by thresholding of a background-subtracted image while searching for a circular-shaped marker within a specific radius range.

3. The system of claim 2, wherein said programming is further configured for classifying the markers based on size, location and color.

4. The system of claim 3, wherein said programming is further configured for tracing the markers over time.

5. The system of claim 4, wherein said programming is further configured for:
for each tracker, determining the corresponding marker from a combination of Euclidian distance and color similarity; and
for all candidates, determining probabilities selecting the marker with the highest probability as the next tracker position.

6. The system of claim 5, wherein said programming is further configured for deterring 3D position from tracker location detected independently in left and right images of the stereo camera.

7. The system of claim 1, wherein said programming is further configured for analyzing a workspace envelope.

8. The system of claim 7, wherein said programming is further configured for transforming tracked 3D hand trajectory into a body-centric coordinate system.

9. The system of claim 8, wherein said programming is further configured for using a body-centric coordinate system to describe the outer envelope of the reached volume.

10. The system of claim 9, wherein said programming is further configured for meshing data to obtain a convex hull.

11. The system of claim 10, wherein said programming is further configured for splitting the mesh data into four quadrants with respect to standardized human body planes.

12. The system of claim 11, wherein the sagittal plane defines the left and right side of the workspace and the horizontal plane defines the top and bottom part of the workspace.

13. The system of claim 12, wherein said programming is further configured for analyzing each quadrant using alpha-shapes and calculating corresponding volume.

14. The system of claim 1, further comprising a remote therapist module configured for communicating with said computer, the therapist module comprising:
(a) a therapist computer with a display;
(b) at least one camera operably coupled to the computer; and
(c) programming executable on the therapist computer configured for communicating with a patient computer, workspace analysis and image rendering.

15. The system of claim 14, further comprising a remote network server module configured for communicating with said computer and the therapist module, the remote network server module comprising:
(a) a computer; and
(b) programming executable on the therapist computer configured for communicating with a patient computer and a therapist computer and data storage.

16. A method for measuring joint angles, range of motion, reachable space, and other motion characteristics of a person, comprising:
acquiring body part motion kinematics in three dimensions from a camera;
measuring bodypart motion trajectories; and
calculating reachable workspace envelope.

17. A method as recited in claim 16, further comprising measuring discrete path lengths of body part movements.

18. A method for measuring joint angles, range of motion, reachable space, and other motion characteristics of a person, comprising:
(a) defining a movement protocol for a body part;
(b) capturing body part trajectories of a subject during performance of the movement protocol for the body part;
(c) fitting trajectories to a workspace template;
(d) transforming fitted trajectories to parameterized coordinates;
(e) determining boundaries from coordinates; and
(f) formulating reachable workspace envelope.

19. A method as recited in claim 18, further comprising segmenting the workspace template into segments.

20. A method as recited in claim 18, further comprising:
calculating reachable workspace surface area; and
calculating reachable workspace volume.
